(19) United States
(12) Patent Application Publication
(10) Pub. No.: US 2026/0108644 A1
(43) Pub. Date: Apr. 23, 2026

(54) AUTOMATED SPIGOT SANITIZATION SYSTEM WITH ROTATING MECHANISM AND UVC STERILIZATION FOR WATER FOUNTAINS

(71) Applicant: Mirza Faizan, Irving, TX (US)

(72) Inventors: Mirza Faizan, Irving, TX (US); Akshara Kommidi, Plano, TX (US); Amreen Syed, Edison, NJ (US); Arjun Kommidi, Plano, TX (US); Jiya Saanvi Singh, Southlake, TX (US); Manha Sadarulanam, Plano, TX (US); Manyatha Simhambhatla, Frisco, TX (US); Meher Saanvi Singh, Southlake, TX (US); Mohammed Kaamil Shadab, Irving, TX (US); Mustafa Farooqui, Irving, TX (US); Rayhan Shanavas, Hyderabad (IN); Riday S. Jogani, Sunnyvale, TX (US); Saanchi Gabri, Frisco, TX (US); Shalaan Umar, Richardson, TX (US); Siddharth Rao, Irving, TX (US); Simran Babaria, Frisco, TX (US); Yusuf Marwat, Allen, TX (US); Zara Majid, Keller, TX (US)

(21) Appl. No.: 18/920,893

(22) Filed: Oct. 19, 2024

(51) Int. Cl. A61L 2/10; E03B 9/20

(52) U.S. Cl. CPC A61L 2/10; E03B 9/20; A61L 2103/00; A61L 2202/122; A61L 2202/14

(57) ABSTRACT

A spigot sanitization system is provided. The system comprises a basin having an upper surface and a lower surface, a set of spigots attachable to the basin, an ultraviolet-C (UVC) sanitization chamber located beneath the lower surface of the basin, a rotating mechanism operatively connected to the set of spigots, a sealing mechanism, and a processor to control, after a passage of a threshold time period, the rotating mechanism to move the first spigot from the upper surface into the UVC sanitization chamber and concurrently move the second spigot from the UVC sanitization chamber to the upper surface for user access, control the UVC sanitization chamber to sanitize the first spigot positioned inside the UVC sanitization chamber while the second spigot is available for the user access, and control the sealing mechanism to stop water flow to the first spigot positioned inside the UVC sanitization chamber.

### TECHNICAL FIELD

This disclosure relates generally to an apparatus for spigot sanitization, and more particularly to an apparatus for automated spigot sanitization using UVC sterilization for water fountains.

### BACKGROUND

Water fountains are commonly used in public spaces such as schools, airports, parks, and offices to provide easily accessible drinking water. However, the spigots of these fountains are frequently exposed to contamination, as they come into contact with users' hands, mouths, and environmental contaminants. This contamination can lead to the spread of harmful bacteria, viruses, and other pathogens, posing a significant health risk to users.

Traditional methods for maintaining hygiene in water fountains rely on manual cleaning, which can be labor-intensive and inconsistent. Additionally, conventional cleaning practices may not eliminate all pathogens, particularly those that accumulate over time or are resistant to basic cleaning procedures. The problem is exacerbated in high-traffic environments where water fountains are used frequently, such as airports and schools, where the risk of contamination is particularly high.

Accordingly, there is a need for an automated system that can sanitize water fountain spigots efficiently and regularly without interrupting their availability to users. Such a system should be adaptable to existing water fountains and provide continuous disinfection without requiring significant manual intervention.

### SUMMARY OF INVENTION

The present invention addresses the above-mentioned challenges by providing an automated spigot sanitization system equipped with a rotating mechanism and a UVC sterilization chamber. The system ensures that one spigot is always available for use while the other is being disinfected. The inclusion of a sealing mechanism prevents water flow to the spigot inside the UVC chamber, preserving the integrity of the sanitization process and reducing water wastage. This system offers an efficient, practical, and cost-effective solution for maintaining spigot hygiene in water fountains, particularly in public and high-traffic environments.

The technical problem lies in ensuring continuous spigot hygiene without disrupting water availability while preventing water flow into the UVC chamber during sanitization.

The disclosed invention provides a system includes a basin with two spigots that alternately dispense water. A motor-driven rotary mechanism moves one spigot into the UVC sanitization chamber for sterilization, while the other remains available for user access. A sealing mechanism controlled by a processor stops water flow to the spigot in the UVC chamber to prevent damage and ensure effective disinfection. Sensors can detect contamination levels, allowing the processor to adjust spigot rotation based on cleanliness. This system ensures ongoing water access while maintaining spigot hygiene through automated UVC sterilization.

In an embodiment, a spigot sanitization system for a water fountain is disclosed. The system comprises a basin having an upper surface and a lower surface, a set of spigots attachable to the basin. The set of spigots includes a first spigot and a second spigot such that the first spigot and the second spigot are configured to alternately dispense water. The system further comprises an ultraviolet-C (UVC) sanitization chamber located beneath the lower surface of the basin. The system further comprises a rotating mechanism operatively connected to the set of spigots. The system further comprises a sealing mechanism configured to stop water flow to one of the set of spigots positioned inside the UVC sanitization chamber. The system further comprises a processor configured to control, after a passage of a threshold time period, the rotating mechanism to move the first spigot from the upper surface into the UVC sanitization chamber and concurrently move the second spigot from the UVC sanitization chamber to the upper surface for user access. The process is further configured to control the UVC sanitization chamber to sanitize the first spigot positioned inside the UVC sanitization chamber while the second spigot is available for the user access. The processor is further configured to control the sealing mechanism to stop water flow to the first spigot positioned inside the UVC sanitization chamber, wherein the sealing mechanism is operable in synchronization with the rotating mechanism to prevent water flow into the UVC sanitization chamber.

In an embodiment, the rotating mechanism comprises a motor-driven rotary actuator configured to rotate the set of spigots in a vertical plane between the upper surface of the basin and the UVC sanitization chamber.

In an embodiment, the sealing mechanism comprises a set of electromagnetic valves operatively connected to the processor.

In an embodiment, each of the set of electromagnetic valves comprises a flow sensor integrated with the corresponding electromagnetic valve, and the flow sensor is configured to detect water flow rate.

In an embodiment, the processor is further configured to determine, based on the detection of the water flow rate by the flow sensor, complete closure of the corresponding electromagnetic valve before the spigot enters the UVC sanitization chamber, and control, based on the determination that the corresponding electromagnetic valve is completely closed, the UVC sanitization chamber to sanitize the first spigot positioned inside the UVC sanitization chamber.

In an embodiment, the UVC sanitization chamber is equipped with UVC light sources arranged to ensure 360-degree sanitization of one of the set of spigots positioned inside the UVC sanitization chamber.

In an embodiment, the UVC sanitization chamber further comprises a temperature sensor configured to detect temperature of the UVC sanitization chamber. The UVC sanitization chamber further comprises a fan. The processor is further configured to control the fan to maintain temperature of the UVC sanitization chamber for effective sanitization. This controlling of the fan is based on the determination that the detected temperature is outside an optimum temperature range.

In an embodiment, the processor is further configured to dynamically adjust the threshold time period based on usage frequency, such that the spigot sanitation cycle occurs more frequently during high usage and less frequently during low usage.

In an embodiment, the UVC sanitization chamber further comprises a drainage mechanism to remove any residual water inside the chamber before sanitization begins, preventing damage to the chamber and optimizing sanitization.

In an embodiment, the processor is further configured to detect spigot contamination levels using one or more sensors and trigger the rotation of the spigots based on the detected contamination level.

In an embodiment, the sealing mechanism includes a pressure-sensitive valve that automatically closes to prevent water flow when the water pressure decreases as the first spigot enters the UVC sanitization chamber.

In an embodiment, the processor is further configured to control a user interface that displays a status of the spigot sanitization process, indicating which spigot is currently in use and which is being sanitized.

In an embodiment, the system further comprises further a solar-powered energy source to provide power to the system.

It is to be understood that both the foregoing general description and the following detailed description are exemplary and explanatory only and are not restrictive of the invention, as claimed.

### BRIEF DESCRIPTION OF THE DRAWINGS

The present application can be best understood by reference to the following description taken in conjunction with the accompanying drawing figures, in which like parts may be referred to by like numerals.

FIG. 1 illustrates a block diagram of a spigot sanitization system, in accordance with an embodiment of the present disclosure.

FIG. 2 illustrates a structural diagram of the spigot sanitization system of FIG. 1, in accordance with an embodiment of the present disclosure.

FIG. 3 illustrates a structural diagram of a motor-driven rotary mechanism in the spigot sanitization system of FIG. 2, in accordance with an embodiment of the present disclosure.

FIG. 4 illustrates a structural diagram of an electromagnetic valve, in accordance with an embodiment of the present disclosure.

### DETAILED DESCRIPTION OF THE DRAWINGS

The following description is presented to enable a person of ordinary skill in the art to make and use the invention and is provided in the context of particular applications and their requirements. Various modifications to the embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention might be practiced without the use of these specific details. In other instances, well-known structures and devices are shown in block diagram form in order not to obscure the description of the invention with unnecessary detail. Thus, the invention is not intended to be limited to the embodiments shown, but is to be accorded the widest scope consistent with the principles and features disclosed herein.

While the invention is described in terms of particular examples and illustrative figures, those of ordinary skill in the art will recognize that the invention is not limited to the examples or figures described. Those skilled in the art will recognize that the operations of the various embodiments may be implemented using hardware, software, firmware, or combinations thereof, as appropriate. For example, some processes can be carried out using processors or other digital circuitry under the control of software, firmware, or hard-wired logic. (The term “logic” herein refers to fixed hardware, programmable logic and/or an appropriate combination thereof, as would be recognized by one skilled in the art to carry out the recited functions.) Software and firmware can be stored on computer-readable storage media. Some other processes can be implemented using analog circuitry, as is well known to one of ordinary skill in the art. Additionally, memory or other storage, as well as communication components, may be employed in embodiments of the invention.

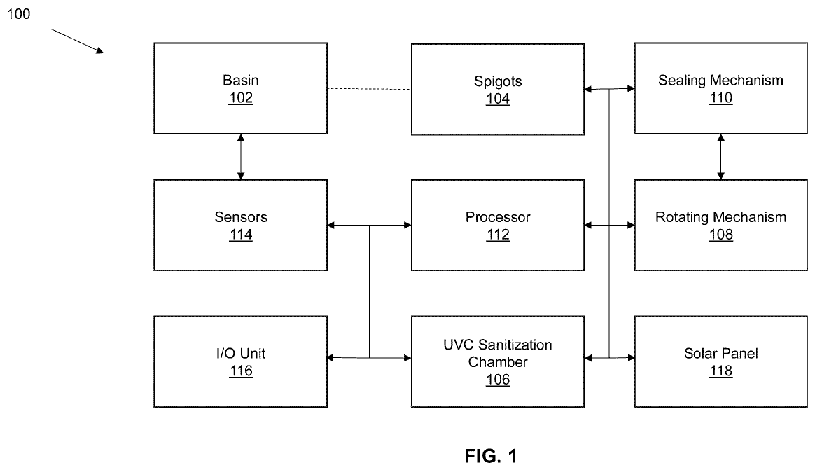

FIG. 1 is a block diagram that illustrates a spigot sanitization system, in accordance with an embodiment of the present disclosure. With reference to FIG. 1, there is shown a spigot sanitization system 100. The spigot sanitization system 100 may include a basin 102, a set of spigots 104, an ultraviolet-C (UVC) sanitization chamber 106, a rotating mechanism 108, a sealing mechanism 110, a processor 112, a set of sensors 114, an input/output (I/O) unit 116, and a solar panel 118.

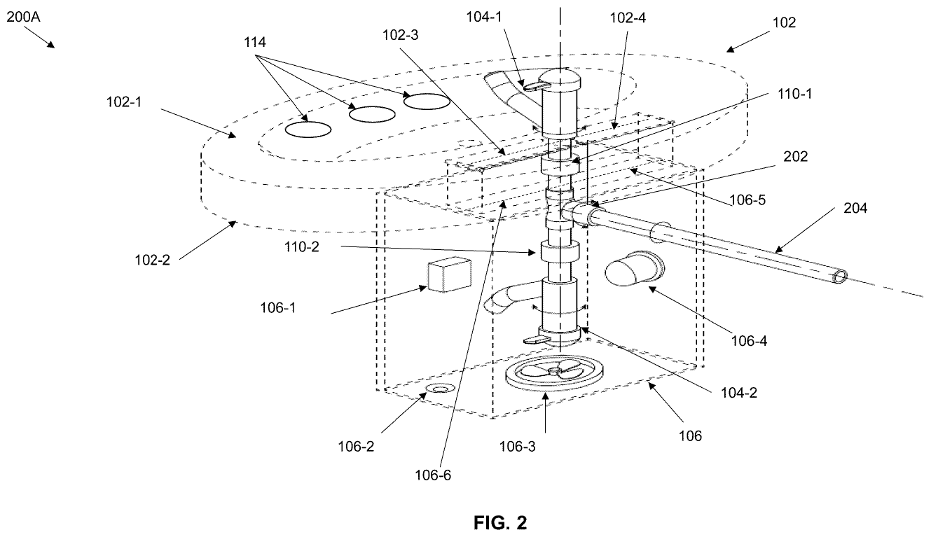

The basin 102 has an upper surface 102-1 for user access and a lower surface 102-2 where critical sanitization components are installed. The basin 102 is capable of collecting water dispensed from one or more spigots. The design of the basin ensures both functionality and ease of integration into existing water fountain setups. In an embodiment, the critical sanitization components installed beneath the lower surface 102-2 of the basin 102 may include the UVC sanitization chamber 106. In an embodiment, the rotating mechanism 108 is also located beneath the lower surface 102-2 such that the lower surface 102-2 supports the UVC sanitization chamber 106 and the rotating mechanism 108. The upper surface 102-1 and the lower surface 102-2 may be viewed in the illustration of FIG. 2.

The basin 102 is designed with an opening or slot that allows the spigots to be alternated between the upper user-facing position and the lower sanitization position. Details on rotation of the spigots between the upper user-facing position and the lower sanitization position are further provided, for example, in FIG. 2.

The set of spigots 104 is attachable to the basin 102. In an example, however not limited to, the set of spigots 104 includes a first spigot 104-1 and a second spigot 104-2. The first spigot 104-1 and the second spigot 104-2 are configured to alternately dispense water. The first spigot 104-1 and the second spigot 104-2 alternate between user-access and sanitization phases. The spigots 104 are connected to the rotating mechanism 108 that allows them to move between the two positions (upper user-facing position and lower sanitization position).

The UVC sanitization chamber 106 is mounted below the lower surface 102-2 of the basin 102, housing the spigot not in use. The UVC sanitization chamber 106 is equipped with UVC light sources positioned around the interior to ensure 360-degree coverage for disinfection. Details on positioning and type of UVC light sources mounted in the UVC sanitization chamber 106 are further provided, for example, in FIG. 2. The UVC sanitization chamber 106 has openings or slots that allow the spigots to rotate in and out of the sanitization position while being shielded from external contamination. Details on rotation of the spigots in and out of the sanitization position are further provided, for example, in FIG. 2.

UVC sanitization is used to disinfect the spigot, ensuring it remains free from bacteria, viruses, and other pathogens that could accumulate through regular use. The UVC sanitization process is effective due to the germicidal properties of Ultraviolet-C (UVC) light, which operates in the 100-280 nanometer (nm) wavelength range. UVC light damages the DNA and RNA of microorganisms, rendering them incapable of reproduction and effectively deactivating them.

In an exemplary scenario, the UVC sanitization procedure includes steps such as spigot rotation, activation of sealing mechanism, UVC light activation, or spigot swap or reset. The steps in the example UVC light activation as mentioned above are not limited to above steps only and may be performed in other possible orders or sequence.

In an example, the spigot rotation includes the use of the rotating mechanism 108 after a pre-determined time period such that the rotating mechanism 108 moves the contaminated spigot from the upper surface of the basin into the UVC sanitization chamber located below the basin.

In an example, the sealing mechanism activation includes the sealing mechanism, such as an electromagnetic valve to block water flow to the spigot being sanitized, preventing water from entering the UVC chamber and protecting the light sources from water damage.

In an example, the UVC light activation is triggered once the spigot is inside the UVC sanitization chamber. UVC light sources (such as UVC LEDs or low-pressure mercury lamps) positioned around the chamber are activated. The UVC light is emitted at wavelengths typically between 250 and 280 nm, which is highly effective in neutralizing microorganisms by disrupting their DNA structure. The UVC sanitization chamber 106 is designed to ensure 360-degree exposure of the spigot to the UVC light. Reflective surfaces inside the chamber can be included to enhance the effectiveness by ensuring that no shadowed areas remain. The sanitization time is calculated to allow for 99.9% germicidal efficiency. This is usually achieved in a matter of seconds to a few minutes, depending on the light intensity.

In an example, the spigot swap and reset includes using the rotating mechanism 108, after the sanitization process is complete, to swap the now-disinfected spigot back to the upper surface for user access while moving the other spigot into the sanitization chamber. This alternating process ensures continuous water availability while maintaining hygienic spigots.

The UVC sanitization chamber 106 and the basin 102 is designed to accommodate the alternating spigot system, allowing the spigots to move in and out of the UVC sanitization chamber 106 and the basin 102 while maintaining a sealed environment during disinfection. The inclusion of an openable slot mechanism ensures that the spigots can transition between the user-facing position and the sanitization position without compromising the effectiveness of the UVC process.

The UVC sanitization chamber 106 is equipped with one or more openable slots through which the spigots enter and exit the chamber. The openable slots ensure that the chamber remains sealed during the sanitization process but can be opened for spigot movement. Details on the openable slots are provided further in the description of FIG. 2. These slots may use a sliding or hinged door mechanism that automatically opens and closes when the spigots are rotated.

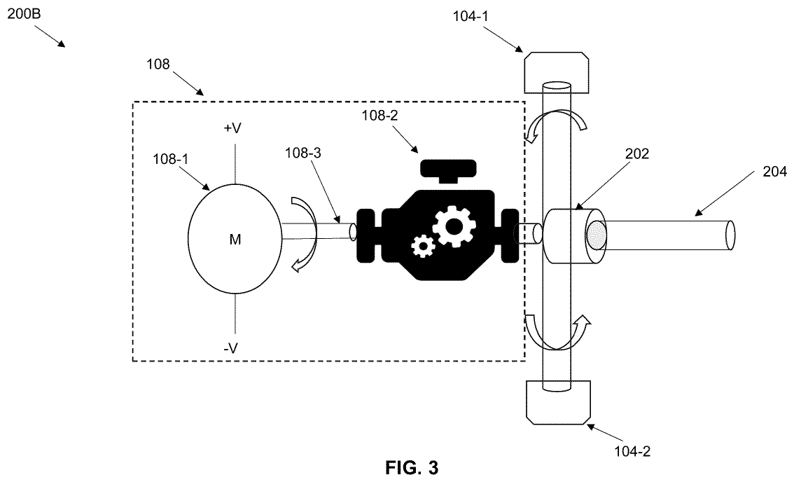

Actuators (such as a motor-driven sliding mechanism or pneumatic system) control the opening and closing of the slot. The actuator operates synchronously with the rotating mechanism, ensuring the slot opens precisely when the spigot is about to move in or out. Details on the actuators controlling the opening/closing of the slot and its synchronization with the rotating mechanism are explained further in the description of FIG. 2 and FIG. 3.

The openable slots are designed to maintain the integrity of the UVC chamber's seal when closed. Gaskets or flexible seals made from UV-resistant materials (e.g., silicone) ensure an airtight closure to prevent UVC light from escaping. The slot mechanism is also designed to prevent dust or contaminants from entering the chamber when the spigots move in or out.

An automatic locking system ensures that the chamber remains securely closed during the sanitization cycle, minimizing the risk of UVC light exposure to the outside environment.

In an additional embodiment, the interior of the chamber is coated with a highly reflective material (such as aluminum or specially coated polymers) to maximize the spread of UVC light and ensure 360-degree sanitization of the spigot.

UVC light sources (such as UVC LEDs or low-pressure mercury lamps) are strategically placed inside the chamber to provide uniform exposure to the spigot. These sources are located on all sides of the chamber to ensure no part of the spigot is left untreated.

In an additional embodiment, the UVC sanitization chamber 106 may include a passive or active cooling system to prevent overheating of the UVC light sources and other components. Passive cooling can be achieved through cooling fins or ventilation slots, while active cooling could use small fans to maintain the optimal operating temperature. These systems are designed to operate without interfering with the spigot movement or the UVC sanitization process.

In an additional embodiment, the UVC sanitization chamber 106 can also be fitted with contamination detection sensors, such as optical or infrared sensors, that assess the cleanliness of the spigots. These sensors can trigger additional sanitization cycles if contamination levels are high.

Additionally, UVC intensity sensors can be installed inside the chamber to monitor the performance of the UVC light sources and ensure they are providing the necessary intensity for effective sanitization.

The entire process of spigot movement into the UVC sanitization chamber 106, activation of the UVC lights, and slot operation is controlled by the processor 112. The processor 112 ensures that the chamber door opens only when necessary and that it remains closed and sealed during the sanitization cycle.

The processor 112 also manages the sanitization cycle length, turning the UVC lights on for a sufficient duration (e.g., 25 minutes) to achieve 99.9% disinfection.

The rotating mechanism 108 is operatively connected to the set of spigots 104. In an example, but not limited to, the rotating mechanism 108 may be a combination of a rotary actuator (motor-driven) for precise spigot rotation, rotating shaft with spigot mounting brackets for secure spigot positioning, processor-controlled movement with safety stops and angle detection via position sensors, and sealed slot mechanism in the basin 102 to guide spigot movement. Additionally, the rotating mechanism 108 may also include a belt and pulley system as an alternative rotation method, or manual override and locking system for redundancy and safety. Details of the rotating mechanism will be more evident in the description of FIG. 3.

The sealing mechanism 110 stops water flow to one of the set of spigots positioned inside the UVC sanitization chamber. The sealing mechanism 110 provides a way to stop water flow to the spigot inside the UVC chamber and ensure water only flows to the spigot available for user access. Examples of the sealing mechanism may include, but not limited to, electromagnetic valves, spring-loaded valves, pneumatic or hydraulic valves, or mechanical flap valves. These mechanisms are integrated into the water flow system and controlled by the processor 112, ensuring synchronization with the spigot rotation and sanitization processes.

In an embodiment, the sealing mechanism 110 includes a pressure-sensitive valve that automatically closes to prevent water flow when the water pressure decreases as the first spigot 104 enters the UVC sanitization chamber 106.

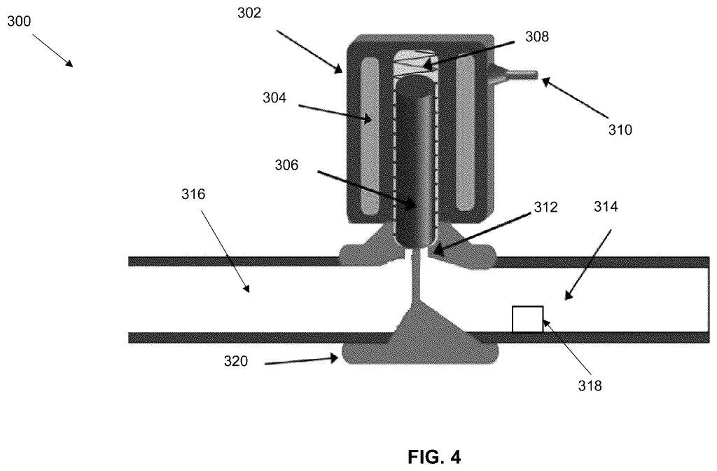

In a preferred embodiment, the electromagnetic valve may be used for precisely stopping water flow to the spigot inside the UVC chamber 106 and ensure water only flows to the spigot available for user access. Details of such an electromagnetic valve are explained further with reference to description of FIG. 4.

The UVC sanitization chamber 106, the rotating mechanism 108, the sealing mechanism 110, and the sensors 114 installed on the basin 102 are operatively connected to the processor 112 that controls, after a passage of a threshold time period, the rotating mechanism 108 to move the first spigot from the upper surface into the UVC sanitization chamber 106 and concurrently move the second spigot from the UVC sanitization chamber 106 to the upper surface for user access. The processor further controls the UVC sanitization chamber 106 to sanitize the first spigot positioned inside the UVC sanitization chamber 106 while the second spigot is available for the user access. The processor 112 further control the sealing mechanism 110 to stop water flow to the first spigot positioned inside the UVC sanitization chamber 106 such that the sealing mechanism 110 is operable in synchronization with the rotating mechanism 108 to prevent water flow into the UVC sanitization chamber 106.

The processor 112 may include suitable logic, circuitry, and interfaces that may be configured to execute program instructions associated with a set of operations to be executed to determine weight distribution, provide the output signal or control the speaker, the display screen, or the haptic device. The processor 112 may include one or more processing units, which may be implemented as an integrated processor or a cluster of processors that perform the functions of the one or more processing units, collectively. The processor 112 may be implemented based on a number of processor technologies known in the art. Example implementations of the processor 112 may include, but are not limited to, an x86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other computing circuits.

The sensors 114 mounted on the basin 102 may be used to detect spigot contamination levels and signals the processor 112 to trigger the rotation of the spigots based on the detected contamination level. Examples of the sensors 114 to detect the contamination level on the spigot may include, but not limited to, optical sensors, bacterial load detection sensors, proximity-based contamination sensors, thermal imaging sensors etc.

In a preferred embodiment, optical sensors may be used to detect the contamination level of the spigots. The sensors 114 may be mounted on the basin, with the most appropriate sensor placed in a strategic position where it can detect the contamination of the spigots. The sensors 114 are connected to the processor 112, which coordinates with the rotating mechanism 108 and the UVC sanitization chamber 106 to maintain the hygiene of the spigots.

The sensors 114 may be housed in waterproof and non-corrosive materials and are optimized to function in the humid environment around water fountains. The data collected by these sensors 114 is processed in real-time, and when the contamination level exceeds a predefined threshold, the processor 112 triggers the rotating mechanism 108 to initiate the sanitization process.

The I/O unit 116 may include a display device that may include suitable logic, circuitry, interfaces, and/or code that may be configured to display a notification for the user based on the output signal from the processor 112. In an embodiment, the processor 112 controls the I/O unit 116 to displays a status of the spigot sanitization process, indicating which spigot is currently in use and which is being sanitized The display device may be realized through several known technologies such as, but not limited to, a Liquid Crystal Display (LCD) display, a Light Emitting Diode (LED) display, a plasma display, and/or an Organic LED (OLED) display technology, and/or other display technologies. In accordance with an embodiment, the display device may refer to a display screen of smart-glass device, a 3D display, a see-through display, a projection-based display, an electro-chromic display, and/or a transparent display.

In an additional embodiment, the I/O unit 116 may also include an input device to provide commands to the processor 112. The user may provide an input to the processor 112 via the I/O unit 116 to activate the sanitization process even before the next scheduled sanitization cycle. In another example, the user may provide input to the processor 112 to adjust the threshold time period based on the user input such that the spigot sanitation cycle occurs more frequently as and when the user needed.

The spigot sanitization system 100 may further include the solar panel 118 which incorporates a solar energy harvesting and power management system that powers the entire sanitization process, including the rotating mechanism 108, the UVC sanitization chamber 106, the sensors 114, the I/O unit 116, and the processor 112. These panels collect sunlight and convert it into electrical energy to power the various components of the sanitization system.

FIG. 2 illustrates a structural diagram of the spigot sanitization system of FIG. 1, in accordance with an embodiment of the present disclosure. With reference to FIG. 2, there is shown a spigot sanitization system 200A. The spigot sanitization system 200A may include a basin 102, a set of spigots 104-1 and 104-2, an ultraviolet-C (UVC) sanitization chamber 106, a sealing mechanism 110, a set of sensors 114, an input/output (I/O) unit 116, and a solar panel 118. The UVC chamber 106 may include a temperature sensor 106-1, a drainage mechanism 106-2, a cooling element 106-3, and a UVC light source 106-4. To ease illustration, the I/O unit 116 and the solar panel 118 of the spigot sanitization system 200A are not shown in FIG. 2 and may be referred to illustration of FIG. 1.

FIG. 3 illustrates a structural diagram of a motor-driven rotary mechanism in the spigot sanitization system of FIG. 2, in accordance with an embodiment of the present disclosure. With reference to FIG. 3, there is shown a portion of a spigot sanitization system 200B. FIG. 3 specifically focuses on the rotating mechanism 108 coupled with the spigots 104-1 and 104-2. In an example, the rotating mechanism 108 may include an actuator 108-1, a gear box 108-2, and a shaft 108-3.

FIG. 2 and FIG. 3 further illustrate a swivel joint connector 202 that is used to ensure a continuous water supply from the water pipe/supply 204 to the spigots (104-1 and 104-2) while allowing them to rotate between their positions. The swivel joint 202 allows the spigots to move freely without causing the water line to become twisted or disconnected. The swivel joint 202 serves as a flexible coupling mechanism between the main water supply line 204 and the rotating spigots 104. It is designed to maintain a watertight connection while enabling the rotational movement of the spigots for sanitization. This type of connector is typically made of durable materials such as brass, stainless steel, or polymer composites to withstand both constant water pressure and mechanical wear.

The processor as described in FIG. 1 is communicatively coupled to the UVC sanitization chamber 106, the rotating mechanism 108, the sealing mechanism 110, the sensors 114, the I/O unit 116, and the solar panel 118. More specifically, FIG. 2 illustrates the components of the rotating mechanism 108, the sealing mechanism 110, and the UVC chamber 106 in co-ordination with each other and the whole system.

In an example, the rotating mechanism comprises a motor-driven rotary actuator such as the actuator 108-1 which is configured to rotate the set of spigots (104-1 and 104-2) in a vertical plane between the upper surface 102-1 of the basin 102 and the UVC sanitization chamber 106 which is beneath the lower surface 102-2 of the basin 102. The actuator 108-1 is coupled to the spigots 104-1 and 104-2 via the gear box 108-2 and the shaft 108-3.

The rotary actuator 108-1 is mounted beneath the lower surface 102-2 of the basin 102 and is operatively coupled to the spigots 104. The actuator is motor-driven and controls the rotational movement of the rotating shaft 108-3 that is connected to the spigots 104. The actuator 108-1 can be an electric stepper motor or a servo motor, capable of precise control over rotational angles. This allows accurate positioning of the spigots 104 into and out of the UVC sanitization chamber 106. The rotational range is limited to a predefined angle, e.g., 180° or 90°, ensuring controlled rotation between the upper and lower surfaces.

The rotating shaft 108-3 connects the spigots 104 to the rotary actuator 108-1 and transfers the rotational force generated by the actuator. The shaft is designed to hold both spigots 104 in a fixed position relative to each other, ensuring that one spigot is always on the upper surface (for user access) while the other is within the UVC sanitization chamber 106. Bushings or bearings are used to provide smooth, low-friction rotation and long-term durability. The rotating shaft 108-3 is also supported by a shaft support structure fixed beneath the basin.

Each spigot is attached to a mounting bracket that is connected to the rotating shaft 108-3. The spigot mounting brackets are positioned symmetrically on either side of the shaft, ensuring that when one spigot is rotated into the chamber, the other spigot moves into the upper surface for user access. The mounting assembly allows the spigots to lock into position on the upper surface of the basin, ensuring a secure water flow when in use.

The processor 112 controls the rotary actuator to ensure timely and synchronized movement of the spigots 104. The processor 112 initiates the rotation of the spigots after a threshold time period (e.g., 25 minutes) and/or based on contamination sensor data. In an additional embodiment, the processor 112 may also dynamically adjust the threshold time period based on usage frequency, such that the spigot sanitation cycle occurs more frequently during high usage and less frequently during low usage. In an example, the usage frequency may be detected by the sensors 114.

The processor 112 also monitors the exact angle of rotation using position sensors (e.g., rotary encoders) attached to the rotary actuator. This ensures precise alignment of the spigots with the basin's upper surface or the UVC sanitization chamber 106.

In an example, where the spigot 104-1 is currently at the upper surface 102-1 for user access and the spigot 104-2 is currently inside the UVC sanitization chamber 106 for sanitization, the clockwise rotational movement of the actuator 108-1 coupled to the spigots via the gear box 108-2 causes the spigot 104-1 to circularly move downward in anti-clockwise direction towards an openable slot 102-3 present in the basin 102 and causes the spigot 104-2 to circularly move upwards towards an openable slot 106-5 in the UVC sanitization chamber 106. The design of the basin 102 and the UVC sanitization chamber 106 is such that the openable slot 102-3 in the basin 102 overlaps or exactly above an openable slot 106-6 in the UVC sanitization chamber 106, and an openable slot 102-4 in the basin 102 overlaps or exactly above the openable slot 106-5 in the UVC sanitization chamber 106. This design ensures smooth hindrance less movement or entry of the spigot 104-1 from the openable slot 102-3 of the basin 102 into the UVC sanitization chamber 106 via the openable slot 106-6. In a similar way, this design also ensures smooth hindrance less movement or entry of the spigot 104-2 from the openable slot 106-5 of the UVC sanitization chamber 106 to the upper surface 102-1 of the basin 102 via the openable slot 102-4.

In an embodiment, when a spigot moves inside the UVC sanitization chamber 106, the supply of water to that spigot must be stopped to ensure that no water is leaked in the chamber. To achieve this purpose, a sealing mechanism is integrated with each spigot. In an example, a sealing mechanism 110-1 is integrated with the water supply of the spigot 104-1 and a sealing mechanism 110-2 is integrated with the water supply of the spigot 104-2.

In an example, the sealing mechanism 110 may include an electromagnetic valve 300 as illustrated in FIG. 4. When a spigot moves inside the UVC sanitization chamber 106, the processor 112 controls the electromagnetic valve 300 to stop water supply to that spigot. The sealing mechanism 110 is operable in synchronization with the rotating mechanism 108 to prevent water flow into the UVC sanitization chamber 106.

Considering the condition that the spigot is moved inside the UVC sanitization chamber 106 and the electromagnetic valve 300 is activated to stop the water supply to that spigot, the processor 112 further controls the UVC sanitization chamber 106 to start the sanitization process which includes switching ON the UVC light source 106-4 and the keeping the UVC light ON for a specific period of time. In a preferred example, the specific period of time must be greater than or equal to 25 minutes.

In an embodiment, when water enters into the UVC sanitization chamber 106, it gets drained out via the drainage 106-2.

In an embodiment, the temperate sensor 106-1 continuously monitors the temperature of the UVC sanitization chamber 106 and supplies this data to the processor 112 on a real time basis. The processor 112 activates the cooling element 106-2 if the temperature of the UVC sanitization chamber 106 goes out of the optimum workable range.

FIG. 4 illustrates a structural diagram of an electromagnetic valve, in accordance with an embodiment of the present disclosure. With reference to FIG. 4, there is shown an electromagnetic valve 300. The electromagnetic valve 300 may include a solenoid 302, a coil winding 304, a plunger or piston 306, a spring 308, a lead wire 310, an orifice 312, an outlet port 314, an inlet port 316, a flow sensor 318, and a valve body 320.

The sealing mechanism 110 may use the electromagnetic valve 300 to control water flow to the spigots 104. These valves are strategically placed in the water supply lines of each spigot. When the processor 112 detects that one spigot needs to be moved into the UVC chamber, it signals the electromagnetic valve 300 to stop water flow to that spigot.

Each spigot is connected to a solenoid-based valve. The valve includes a solenoid 302 that, when energized, activates a plunger or piston 306 to either open or close the water flow to the spigot. The valve can be a normally closed valve, meaning the default state of the valve is closed, and it only opens when the solenoid 302 is energized.

Two water supply lines are connected to the first and second spigots, respectively. The electromagnetic valves are integrated into these lines. The electromagnetic valve 300 is controlled by a circuit connected to the processor 112. The processor 112 sends an electrical signal to energize or de-energize the solenoid 302, thus controlling the valve's opening and closing.

When the first spigot is in the UVC chamber, the processor signals the electromagnetic valve corresponding to the first spigot to close, thereby blocking water flow. Simultaneously, it signals the valve corresponding to the second spigot (on the upper surface) to open, allowing water to flow through that spigot.

After the sanitization cycle, the roles of the valves are reversed as the second spigot moves to the UVC chamber and the first spigot moves to the upper surface.

The electromagnetic valves 300 are made of corrosion-resistant materials, such as stainless steel for the valve body and Teflon or rubber for internal sealing components to handle water pressure and prevent leakage.

In an embodiment, a flow sensor 318 is integrated with each of the electromagnetic valves 300, and the flow sensor 318 is configured to detect water flow rate in the outlet port 314. Based on a result of the detection of the water flow rate by the flow sensor 318, the processor 112 determines that the electromagnetic valve 300 has completely blocked the water supply to the corresponding spigot before that spigot enters the UVC sanitization chamber. In an example, when the flow sensor 318 detects that the water flow rate in the outlet port 314 is zero or less than a threshold value, the processor 112 records this as an indication that the water in the corresponding spigot is blocked and that spigot is ready to enter the UVC sanitization chamber 106. Based on the determination that the corresponding electromagnetic valve is completely closed or the indication that the water in the corresponding spigot is blocked, the processor 112 controls the UVC sanitization chamber to sanitize the spigot positioned inside the UVC sanitization chamber 106.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

What is claimed is:

1. A spigot sanitization system for a water fountain, comprising:
a basin having an upper surface and a lower surface;
a set of spigots attachable to the basin, wherein
the set of spigots includes a first spigot and a second spigot, and
the first spigot and the second spigot are configured to alternately dispense water;
an ultraviolet-C (UVC) sanitization chamber located beneath the lower surface of the basin;
a rotating mechanism operatively connected to the set of spigots;
a sealing mechanism configured to stop water flow to one of the set of spigots positioned inside the UVC sanitization chamber; and
a processor configured to:
control, after a passage of a threshold time period, the rotating mechanism to move the first spigot from the upper surface into the UVC sanitization chamber and concurrently move the second spigot from the UVC sanitization chamber to the upper surface for user access;
control the UVC sanitization chamber to sanitize the first spigot positioned inside the UVC sanitization chamber while the second spigot is available for the user access; and
control the sealing mechanism to stop water flow to the first spigot positioned inside the UVC sanitization chamber, wherein the sealing mechanism is operable in synchronization with the rotating mechanism to prevent water flow into the UVC sanitization chamber.

2. The system of claim 1, wherein the rotating mechanism comprises a motor-driven rotary actuator configured to rotate the set of spigots in a vertical plane between the upper surface of the basin and the UVC sanitization chamber.

3. The system of claim 1, wherein the sealing mechanism comprises a set of electromagnetic valves operatively connected to the processor.

4. The system of claim 3, wherein
each of the set of electromagnetic valves comprises a flow sensor integrated with the corresponding electromagnetic valve, and
the flow sensor is configured to detect water flow rate.

5. The system of claim 4, wherein the processor is further configured to:
determine, based on the detection of the water flow rate by the flow sensor, complete closure of the corresponding electromagnetic valve before the spigot enters the UVC sanitization chamber, and
control, based on the determination that the corresponding electromagnetic valve is completely closed, the UVC sanitization chamber to sanitize the first spigot positioned inside the UVC sanitization chamber.

6. The system of claim 1, wherein the UVC sanitization chamber is equipped with UVC light sources arranged to ensure 360-degree sanitization of one of the set of spigots positioned inside the UVC sanitization chamber.

7. The system of claim 1, wherein
the UVC sanitization chamber further comprises a temperature sensor configured to detect temperature of the UVC sanitization chamber,
the UVC sanitization chamber further comprises a fan,
based on the detected temperature is outside an optimum temperature range, the processor is further configured to control the fan to maintain temperature of the UVC sanitization chamber for effective sanitization.

8. The system of claim 1, wherein the processor is further configured to dynamically adjust the threshold time period based on usage frequency, such that the spigot sanitation cycle occurs more frequently during high usage and less frequently during low usage.

9. The system of claim 1, wherein the UVC sanitization chamber further comprises a drainage mechanism to remove any residual water inside the chamber before sanitization begins, preventing damage to the chamber and optimizing sanitization.

10. The system of claim 1, wherein the processor is further configured to detect spigot contamination levels using one or more sensors and trigger the rotation of the spigots based on the detected contamination level.

11. The system of claim 1, wherein the sealing mechanism includes a pressure-sensitive valve that automatically closes to prevent water flow when the water pressure decreases as the first spigot enters the UVC sanitization chamber.

12. The system of claim 1, wherein the processor is further configured to control a user interface that displays a status of the spigot sanitization process, indicating which spigot is currently in use and which is being sanitized.

13. The system of claim 1, further comprising a solar-powered energy source to provide power to the system.
